(19) United States
(12) Patent Application Publication
(10) Pub. No.: US 2008/0280340 A1
(43) Pub. Date: Nov. 13, 2008

(54) Method and Apparatus for Bacterial Transformation by Electroporation with Waveforms Incorporating Pulsed Rf Between 3 and 125 Mhz

(76) Inventors: Mikhail V. Tyurin, Lebanon, NH (US); Lee R. Lynd, Meriden, NH (US); Charles R. Sullivan, West Lebanon, NH (US)

Correspondence Address: LATHROP & GAGE LC, 4845 PEARL EAST CIRCLE, SUITE 300, BOULDER, CO, 80301, US

(21) Appl. No.: 11/579,640

(22) PCT Filed: May 6, 2005
(86) PCT No.: PCT/US05/16205

§ 371 Date: May 14, 2008

Related U.S. Application Data
(60) Provisional application No. 60/568,756, filed on May 6, 2004.

(51) Int. Cl. C12N 13/00; C12M 1/00

(52) U.S. Cl. 435/173.6; 435/285.2

(57) ABSTRACT

A method and apparatus for electroporation includes placing a mixture of bacterial suspension and transforming DNA into an electroporation cuvette. The resulting sample is subjected through a current-limiting device to a complex 5 waveform including a burst of high-voltage radio-frequency current, which in some embodiments is superimposed on a biphasic high-voltage DC pulse, and in other embodiments on a high-voltage lower-frequency AC burst. The total waveform has at least an initial portion greater than eleven thousand volts per centimeter of electrode spacing, and a later portion in some embodiments is reduced to less than thirty percent 10 of magnitude of the initial portion. Transformed bacteria are selected by culture in selective medium in an embodiment. The high-voltage radio-frequency current is between 3 and 125 MHz, and in an embodiment is 24 MHz.

### RELATED APPLICATIONS

This application claims priority to U.S. patent application 60/568,756, filed May 6, 2004, entitled “USE OF INDUCED OSCILLATIONS TO ACHIEVE HIGH EFFICIENCY TRANSFORMATION OF DIFFICULT TO TRANSFORM BACTERIA” the disclosure of which is incorporated herein by reference.

### US GOVERNMENT RIGHTS

The United States Government has certain rights in this invention pursuant to contract Phase I SBIR DE-FG02-03ER83593 awarded by the Department of Energy.

### FIELD OF THE INVENTION

The present document relates to methods and apparatus for engineering genomes of microorganisms. In particular, the present document relates to transformation of bacteria by driving DNA segments across a bacterial cell membrane with an electric field.

### BACKGROUND OF THE INVENTION

### Engineering Bacterial Genomes

Bacteria typically have genomes including one or a few chromosomal strands of Deoxyribose Nucleic Acid (DNA) having thousands of segments known as genes. Each gene includes a sequence of nucleotides that code for one or more peptides, or proteins, together with regulatory nucleotide sequences such as promoters, start codons, and stop codons. Bacteria may also incorporate shorter DNA segments such as plasmids and dormant bacteriophages, which may also contain genes and which may reside in the cytoplasm alone or be incorporated into the bacterial genome.

Typical bacterial species encode proteins that have evolved according to the needs of the species in the environment it normally inhabits. These proteins typically include proteins for reproduction of the bacterial cell, for energy production, for producing fundamental building blocks of the cell like nucleotides, for producing motility structures like flagella, and for toxins that give that species a survival advantage over other species in the same environment.

Bacteria have been engineered to produce proteins unnecessary for survival of that species, but of interest to humans. This has been done by isolating or creating a new segment of DNA encoding a desired protein and inserting the new segment into the bacterial genome; once inserted the new segment reproduces with the bacteria and, if properly designed and inserted, may produce the desired protein. The process of inserting the new DNA segment is known as transformation.

Similarly, transformation can be used to disable selected genes normally present in the bacterial genome, or to increase production of preferred products.

Alteration of bacterial genomes can be of use in adapting an organism to survive in a different environment, or to modify the organism's metabolic pathways to produce non-protein metabolic products of interest to humans.

### Electroporation

Electroporation is a term describing transport of hydrophilic molecules across a hydrophobic membrane via electrically formed pores (electropores).

While some bacteria can be transformed simply by adding a solution of new DNA to culture media; most bacteria require further manipulation to transport the new DNA across the bacteria's cell wall and membrane into the interior of the bacterial cell. One such technique is Electroporation.

DNA generally has a negative charge in aqueous solution; DNA is an acid and liberates hydrogen ion in solution at physiological pH. DNA therefore tends to move towards a positively charged electrode when an electric field is applied to a DNA solution, a phenomenon known as electrophoresis.

A desired new DNA plasmid is prepared in aqueous solution of low ionic strength, and added to bacterial cells suspended in an electroporation buffer. The mixture is typically kept on ice to prevent DNA degradation and to avoid overheating the bacteria during electroporation. Electroporation is performed by exposing the mixture of DNA solution and suspended bacteria to a high-intensity, brief, electric field. The intense field carries DNA molecules across the hydrophilic cell wall by electrophoresis. The intense field also carries DNA molecules through a temporary electropore in the hydrophobic cell membrane into some, but far from all, of the bacteria.

The desired new DNA plasmid may include sequences homologous to portions of the bacterial chromosome at which they can insert into the bacterial chromosome. The new plasmid may alternatively include portions that code for integrases that incorporate portions of the plasmid into the bacterial chromosome. The new plasmid may be capable of surviving and replicating within the bacteria.

The bacteria are then cultured under conditions favorable to growth of bacteria incorporating the desired new DNA. Typically a gene encoding for resistance to an antibiotic is included in the new DNA, and that same antibiotic is included in a post-electroporation culture media. Transformed bacteria are selected for because they have a survival advantage over untransformed bacteria in this media.

Typically, electroporation is performed by placing the bacterial suspension and the transforming DNA between electrodes of a chilled electroporation cuvette and applying an electric pulse to the cuvette. A high, DC, or RF modulated DC voltage pulse is applied to the electrodes for a preset time typically up to several dozen milliseconds.

### Difficult to Transform Bacteria.

Some bacteria with complex intracellular morphology and/or complex cell development cycle are known as “difficult to electrotransform”. When transforming these bacteria, it is important to identify the right growth medium, specific growth stage of the culture, and some other biological conditions, such that the cells become as “electrocompetent” as possible before performing electroporation.

When bacteria of some species are subjected to stressful conditions, they may form hardy endospores. Endospores are generally smaller than normal vegetative cells, and much more resistant to chemical, thermal, dessication, and other environmental hazards than normal vegetative cells. Electroporation may provide sufficient chemical, electrical, and thermal stress to trigger spore formation in some bacteria. As spores form, much of the cellular contents, often including the newly inserted and desired DNA plasmid if sporulation occurs immediately after electroporation, is excluded from the spore. Spore forming bacteria with complex lifecycles therefore are often difficult to transform.

Some bacteria, such as Acinetobacter species, have intracellular granules or vesicles that can block inserted DNA plasmids from the nucleoid region of the cell where the genome and DNA replication enzymes are located. These bacteria can also be difficult to transform because only DNA inserted into the proper part of the cell is likely to be expressed, and because these granules and vesicles may block plasmids from reaching the proper part of the cell.

Clostridium is a genus of Gram-positive, spore-forming, anaerobic, bacteria, including several species that are difficult to transform, typically derive energy through fermentation and for which free oxygen is toxic. Example members of the genus include Clostridium perfringens, pathogenic in animals and man; Clostridium botulinum, noted for production of potent toxins; as well as Clostridium thermocellum, capable of fermenting cellulose at 60° C.

Prior techniques for transformation of difficult-to-transform bacteria have included modifying the bacterial cell walls by growing the bacteria in media containing ingredients that damage developing cell walls, or by partially digesting the cell walls. The weakened cell walls then allow desired DNA plasmids to reach the cell membrane more rapidly, so that plasmids are more likely to pass through electropores into the cells. It has been found that weakening cell walls often adversely affect viability of the bacteria, viability is often so poor that electroporation yield remains unacceptably low.

It is desirable to improve transformation yield of difficult-to-transform bacteria to expedite research performed with such bacteria.

### Selection of Transformed Bacteria

Once electroporation is performed, transformed bacteria may be selected by culturing the bacteria in a selective media. Selective medium contains at least one antibiotic for which a gene of resistance is included in the desired DNA plasmid. In such media, only transformed bacteria thrive.

Alternatively, electroporated bacteria may be cultured into colonies on an agar plate and bacterial products blotted onto a membrane. The membrane can then be stained with fluorescent antibodies to proteins encoded on the desired DNA plasmid. Colonies expressing those proteins will then have associated fluorescent marks on the membrane, thereby allowing identification of colonies that express those proteins.

Modified Clostridium thermocellum May Help Produce Biofuels

Most plants produce sugars by photosynthesis in abundance. Typical plants process most of the sugars they produce into cellulose, forming much of the cell wall and supporting fiber of angiosperms. Only a small proportion of sugars become starches in seed.

There are many uses for the seed of corn, wheat, oats, or other grains, both for food, animal feed, and for fermentation into alcohols. Many organisms, including humans, produce amylase enzymes capable of hydrolyzing starch.

Mammals, yeast, and other eukaryotic organisms lack enzymes for hydrolyzing cellulose; sugars linked with beta-glucoside bonds in cellulose are not well used and often become waste. Grass, wood, agricultural residues (including cornstalks, wheat and oat straw, and manure), and municipal solid waste (paper) have high cellulose content.

Clostridium thermocellum has the ability to hydrolyze cellulose, it ferments the resulting sugars into a mixture of alcohols and organic acids, including acetic acid.

It has been proposed that a strain of Clostridium thermocellum suitable for use in industrial production of ethanol from cellulose can be more easily engineered if this organism's resistance to transformation by electroporation can be overcome since multiple, substantial, modifications of its genome are required. In particular, it is desirable to decrease organic acid production and increase both ethanol production and ethanol tolerance.

### SUMMARY

A method of electroporation includes placing a mixture of bacterial suspension and transforming DNA into an electroporation cuvette. The resulting sample is subjected through a current-limiting resistor to a complex waveform including a burst of high-voltage radio-frequency current, which in some embodiments is superimposed on a biphasic high-voltage DC pulse. The total waveform has at least an initial portion greater than eleven thousand volts per centimeter of electrode spacing. In some embodiments the waveform in a later portion is reduced to between ten and thirty percent of the magnitude of the initial portion. Transformed bacteria are selected by culture in selective medium.

### BRIEF DESCRIPTION OF THE FIGURES

FIG. 1 is a block diagram of a prior-art electroporation apparatus.

FIG. 2 illustrates the principle of electroporation.

FIG. 3 is a block diagram of the present electroporation apparatus.

FIG. 4 is an illustration of DC and AC components of the high-voltage burst.

FIG. 5 is an illustration of DC and AC components of an alternative high-voltage burst.

FIG. 6 is an abbreviated flow chart of a method of bacterial transformation.

FIG. 7 is an illustration of DC and AC components of an alternative high-voltage burst.

FIG. 8 is an illustration of a composite-AC high-voltage burst.

### DETAILED DESCRIPTION OF THE EMBODIMENTS

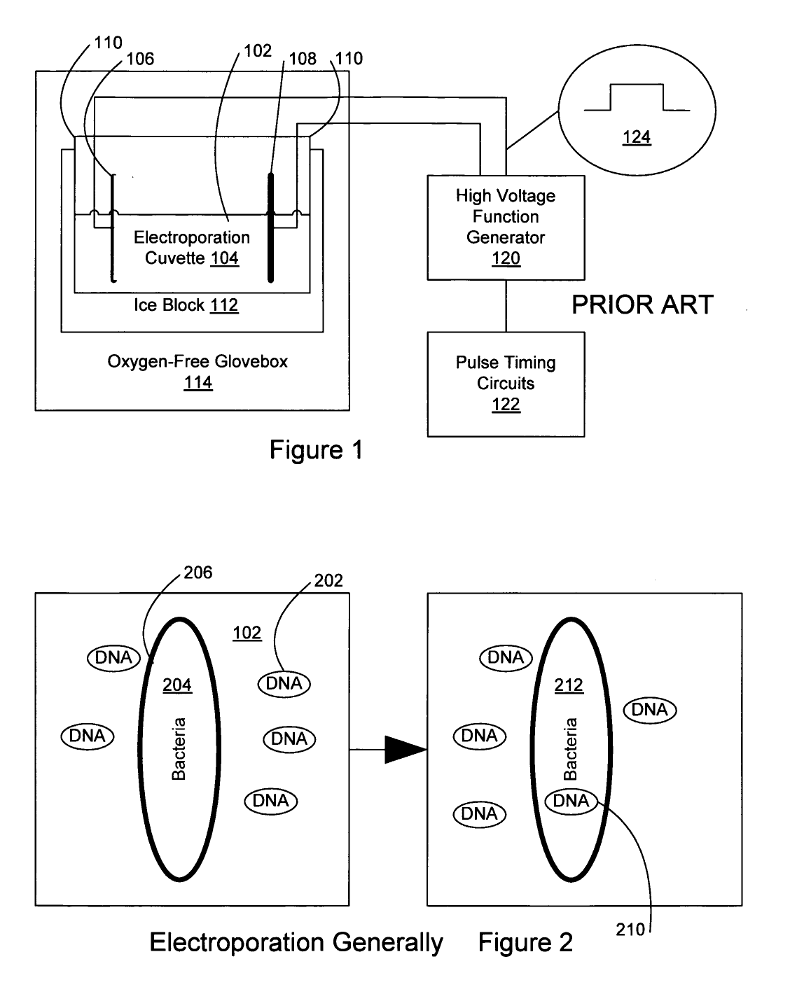

FIG. 1 illustrates prior-art electroporation apparatus similar to that described in Tyurin M. V., Padda R., Ke-xue Huang, Wardwell S., Caprette D., Bennett G. N. (2000): Electrotransformation of Clostridium acetobutylicum ATCC 824 Using High-Voltage Radio Frequency Modulated Square Pulses//Journal of Applied Microbiology. 88(2): 220-227 (hereinafter Tyurin, 2000), the text of which is hereby incorporated by reference. A suspension of cultured bacteria in a solution 102 of desired-sequence DNA is placed in an electroporation cuvette 104. Care is taken to ensure that the solution 102 is as free of salt as possible, nonionic solutes are used to provide appropriate osmolarity. The cuvette 104 has a first electrode 106 and a second electrode 108 spaced both exposed to the solution 102 but electrically insulated by a polypropylene microcentrifuge tube 110 from a surrounding ice block 112. When working with anaerobic bacteria, the entire cuvette 104 and ice-block 112 are placed in an oxygen-free glovebox 114. The first 106 and second 108 electrodes are connected to the output of a high-voltage electrical function generator 120, or operating under control of pulse timing circuits 122.

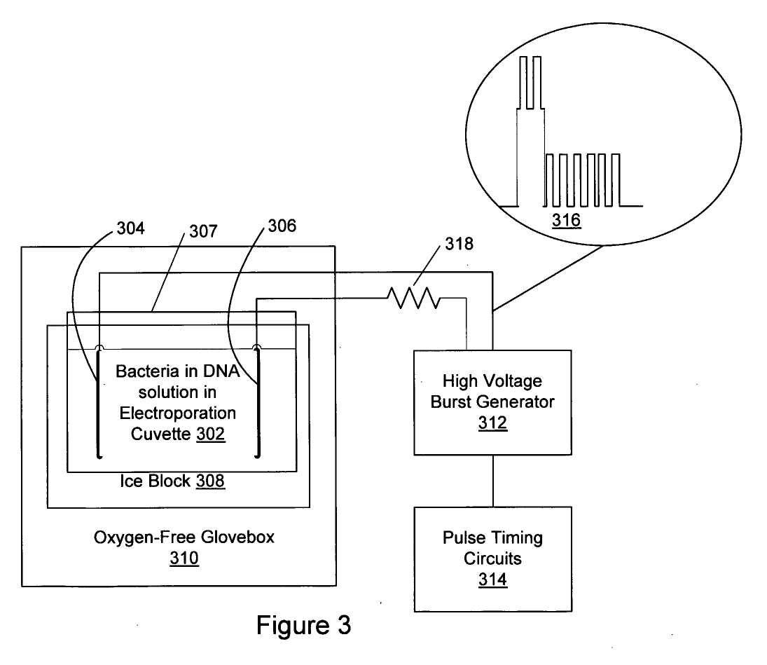

Reported apparatus includes high-voltage electrical function generators 120 having direct current DC output, and high-voltage electrical function generators having a one-hundred kilohertz AC burst superimposed on a DC pulse. In particular, Tyurin 2000 in his FIG. 3 taught that better results were obtained with one-hundred kilohertz AC superimposed on a DC pulse than with higher frequencies in the range one hundred fifty to three hundred fifty kilohertz.

When the prior apparatus of FIG. 1 is used, the operator triggers the pulse timing circuits 122, and the pulse generator provides either a square pulse as indicated 124, an exponentially decaying pulse, or a superposition of a high-voltage DC pulse with one hundred kilohertz AC of amplitude one-tenth to one-third the magnitude of the DC pulse between the first 106 and second 108 electrodes of the cuvette. While DC pulses are standard in the art, Tyurin 2000 disclosed transformation of Clostridium species using a pulse having an AC signal of 100 kHz superimposed on a DC pulse. High-voltage pulses above one kilovolt are typically used.

The high-voltage electric field applied between the electrodes 106, 108, causes current to flow through the solution 102 of desired DNA plasmids 202 (FIG. 2) with suspended bacteria 204. DNA plasmids 202 are typically circular of DNA with a replication initiation site. The current, which readily flows through the saline intracellular fluid, burns minute holes in the bacterial cell walls 206, and electrophoretically shifts DNA molecules in the solution, through the cell walls, and some molecules may shift through the minute holes. After the high-voltage current pulse, some desired DNA molecules 210 end up inside transformed 212 bacteria, where the desired DNA molecules 210 may be expressed and further incorporate into the bacterial genome.

In the present electroporation apparatus as illustrated in FIG. 3, a suspension of cultured bacteria in a solution of desired-sequence DNA is placed in an electroporation cuvette 302. The desired-sequence DNA is preferably in the form of a plasmid. The cuvette has first 304 and second 306 polished parallel-plate stainless-steel electrodes spaced approximately two millimeters (distance D) apart in a two-milliliter polypropylene disposable centrifuge tube 307, and both the electrodes and cuvette contents are electrically insulated from, but thermally coupled to, a surrounding ice block 308. In doing so, part of the tube 307 is placed within a cavity in the ice block 308. When working with anaerobic bacteria such as Clostridium thermocellum, the cuvette 302 and icewater bath 308 are kept in an oxygen-free glovebox 310. The cuvette's electrodes 304, 306 are connected to a high-voltage burst generator 312, controlled by a pulse generator 314.

When the present electroporation apparatus is operated, an operator triggers the pulse generator 314 to fire the high-voltage burst generator 312. The high-voltage burst generator then provides a high-voltage burst 316 through a current limiting resistor 318.

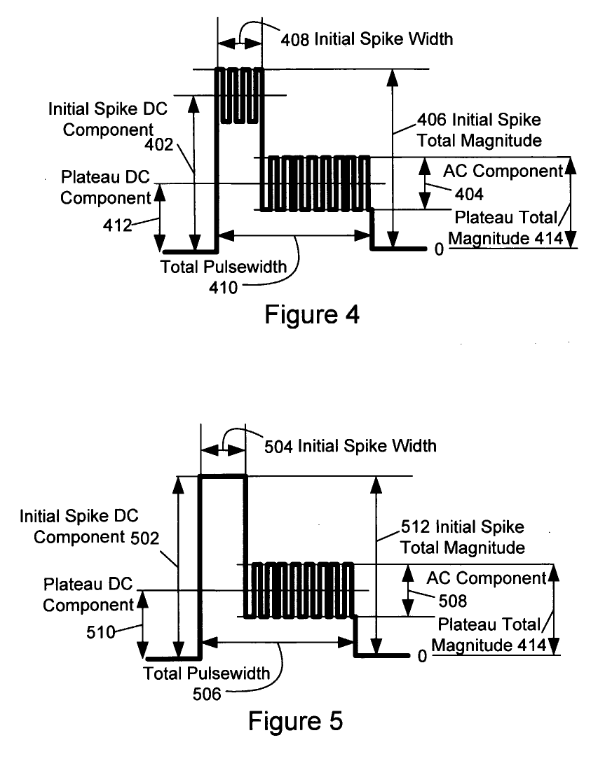

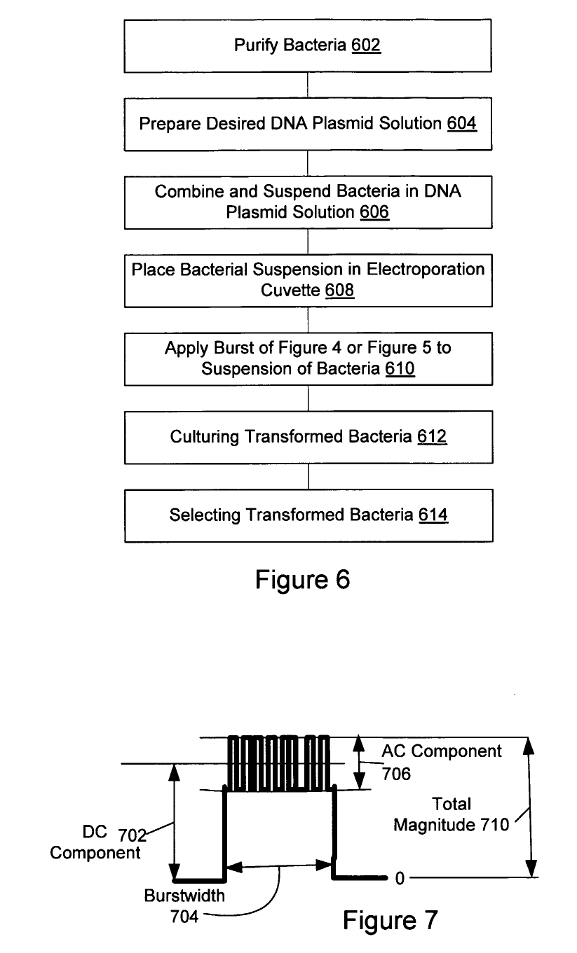

The high-voltage burst 316 can be described as illustrated in FIG. 4 as the superposition of a high-voltage Initial Spike of Direct Current (DC) pulse having a DC component amplitude 402 with a superimposed sinusoidal high radio-frequency Alternating Current (AC) peak-to-peak amplitude 404 such that the Initial Spike Total Amplitude 406 is in the range of from 12 to 25 kilovolts per centimeter of electrode spacing D. The electroporation pulse has an initial spike pulsewidth 408 between approximately five and twenty percent, and in an embodiment is about ten percent of the total pulsewidth 410 of initial spike and plateau; the total pulsewidth 410 ranges from three to twenty-five milliseconds, and is preferably from eight to ten milliseconds. The plateau therefore is greater in length than half of the total pulsewidth 410. The plateau continues the radio-frequency alternating current at approximately the same peak-to-peak amplitude 404 as during the initial spike, and has a plateau DC component 412 that is between ten and thirty percent of the initial spike DC component, and has magnitude greater than half of the AC peak-to-peak amplitude. The total magnitude 414 of the plateau of the voltage burst is the sum of the DC component 412 plus half the AC peak-to-peak amplitude 404. The sinusoidal AC component has a frequency of between 3 and 125 MHz, being preferably within twenty percent of 24 MHz, and has peak-to-peak magnitude greater than six and a half percent of the initial spike total amplitude 406. The high-voltage burst may be of either polarity, since relative voltages between the electrodes 304, 306 induce current flow in the electroporation cell. The composite of AC and DC components is known herein as a high-voltage burst.

Since in the embodiment of FIG. 4, the AC component begins with the initial spike, the AC component overlaps the initial spike.

It is believed that the initial spike serves to create pores in the bacterial cell membrane and start discharge through the cuvette, while the plateau portion serves to electrophorese the desired DNA plasmid through cell wall and through the pores into cells. It is also believed that the AC component causes current paths through the bacterial sample to vary, such that cell damage is spread out and not focused on any one portion of the cell.

Pulses approximating that described in FIG. 4, such as those with a rapid exponential decay from the initial spike DC component to the plateau level, are expected to produce similar results.

It is expected that with certain bacteria, especially difficult to transform members of genus Clostridium, the present electroporator using the waveform of FIG. 4 will give substantially better yields of transformed bacteria than achieved by other workers and with other apparatus. It was also found that a greater percentage of bacteria appeared largely intact after electroporation than with conventional electroporation.

The present electroporator is also expected to be successful with bacterial strains from species of Thermoanaerobacterium and Thermoanaerbacter, and other bacteria having characteristics resembling those of Clostridium species. It is also expected that the present electroporator will be successful with Acinetobacter.

Experiments where the AC component was lacking show significantly reduced transformation efficiency.

An alternative high-voltage burst is illustrated in FIG. 5 as the superposition of a high-voltage Initial Spike of Direct Current (DC) pulse having a DC component amplitude 502 in the range of from 12 to 24 kilovolts per centimeter of electrode spacing D, or 2400 to 4800 volts for electrodes spaced two millimeters apart. The electroporation pulse has an initial spike pulsewidth 504 approximately ten percent of the total pulsewidth 506 of initial spike and plateau; the total pulsewidth 506 ranges from three to twenty-five milliseconds, and is preferably from eight to ten milliseconds. The plateau has superimposed a sinusoidal radio-frequency alternating current (AC) 508 component, and has a plateau DC component 510 that is between ten and thirty percent of the initial spike DC component, and has magnitude greater than half of the AC peak-to-peak amplitude 508. The total magnitude 512 of the voltage burst is the sum of the DC component 510 plus half the AC peak-to-peak amplitude 508. The AC component has a frequency of between 3 and 125 MHz, being preferably about 24 MHz, and has a peak-to-peak magnitude greater than six and a half percent of the peak amplitude of the initial spike 502.

In the embodiment of FIG. 5, the AC component begins as the initial spike ends.

In summary, the process of generating transformed bacteria begins by purifying a culture of bacteria 602 to remove most contaminating salts from the culture media. A solution of desired DNA plasmids is prepared 604, this is combined 606 with the purified bacteria to produce a suspension of bacteria in a solution of DNA plasmids. The bacteria may be suspended in salt-free solvent and DNA solution added, or the bacteria may be suspended directly in the DNA solution.

The suspension of bacteria in DNA solution is placed 608 in the electroporation cuvette 302 such that the electrodes 304, 306 make contact with the solution. A burst as described above with reference to FIG. 4 or FIG. 5 is applied 610 to the suspension. Surviving bacteria are cultured 612 and selected 614 to provide a culture of transformed bacteria.

The culturing 612 and selection 614 are in a first embodiment performed by including an antibiotic resistance gene in the DNA plasmids, and including the related antibiotic in culture media in which surviving bacteria are cultured 612.

In an alternate embodiment selection 614 is performed by blotting.

FIG. 7 illustrates an alternative burst that has been successfully used to transform Clostridium thermocellum bacteria. The burst is essentially the superposition of a square DC pulse represented as DC component 702 having a pulsewidth 704 of between three and twenty-five milliseconds, in an embodiment the pulsewidth is between eight and twelve milliseconds. The DC component 702 is superimposed on an AC component 706 having peak-to-peak magnitude greater than six and a half percent of the magnitude of the DC component 702, and in an embodiment a frequency of twenty-four megahertz. A total magnitude 710 of the voltage burst is the sum of the DC component 702 plus half the AC peak-to-peak amplitude 706. The AC component has a frequency of between 3 and 125 MHz, being preferably within twenty percent of 24 MHz. The burst of FIG. 7 has total magnitude 710 in the range of between eleven kilovolts and twenty-four kilovolts per centimeter of electrode spacing in the electroporation cuvette.

It was found that, with difficult to transform members of genus Clostridium, the present electroporator using the waveform of FIG. 7 gave better yields of transformed bacteria than achieved by other workers and with other apparatus. It was also found that a greater percentage of bacteria appeared largely intact after electroporation than with conventional electroporation.

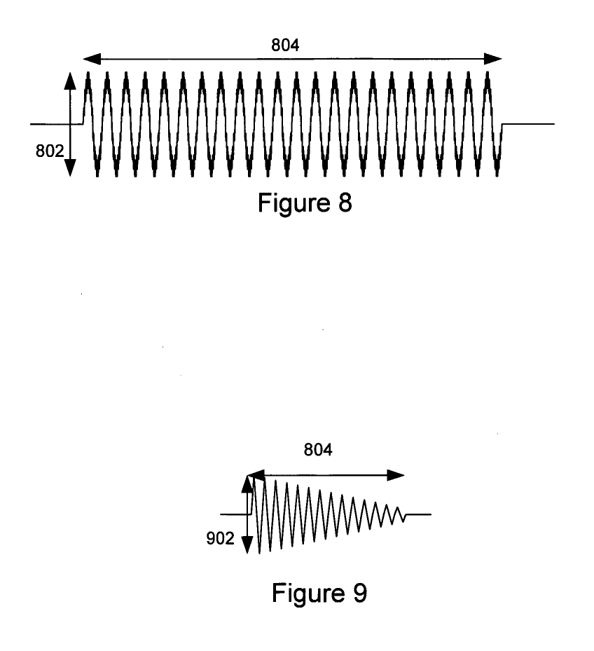

In an alternative embodiment, illustrated in FIG. 8, a burst having a first and a second superimposed AC signals with combined zero-to-peak magnitude 802 reaching between eleven kilovolts and twenty-four kilovolts per centimeter of spacing between electrodes in the electroporation cuvette. The first AC signal has a frequency of between twenty and five hundred kilohertz, and preferably about one hundred kilohertz, the second AC signal has a frequency of between three and one hundred twenty-five, and preferably about twenty-four, megahertz. The second AC signal has a magnitude between six and thirty percent of the magnitude of the first AC signal. The burst has a width 804 of between three and twenty-five milliseconds, in an embodiment the width is between eight and twelve milliseconds.

In yet another alternative embodiment, illustrated in FIG. 9, a damped AC burst having a center frequency of between three and one hundred twenty-five, and preferably about twenty-four, megahertz. The damped burst has an initial zero-to-peak magnitude 902 reaching between eleven kilovolts and twenty-four kilovolts per centimeter of spacing between electrodes in the electroporation cuvette. The burst tapers in amplitude to a level approximately ten percent of the initial magnitude over a width 904 of between three and twenty-five milliseconds, in an embodiment the width is between eight and twelve milliseconds.

It is anticipated that the current limiting resistor 318 may be replaced by other forms of current limiting devices appropriate for the waveform applied. In particular, it is expected that a reactive component such as an inductor, or a reactive network incorporating one or more inductors, resistors, and capacitors, are particularly suitable for use as a current limiting device with waveforms incorporating the AC components herein described. In some embodiments, current sensing with current limiting through active feedback may also be used.

With the use of substantial AC components in the transforming pulse as herein described, it is anticipated that some embodiments may embed the current limiting device into an RF or pulse transformer that serves to increase voltage between the burst generator and the cuvette.

While the invention has been particularly shown and described with reference to a preferred embodiment thereof, it will be understood by those skilled in the art that various other changes in the form and details may be made without departing from the spirit and scope of the invention. It is to be understood that various changes may be made in adapting the invention to different embodiments without departing from the broader inventive concepts disclosed herein and comprehended by the claims that follow.

1. A method for transforming bacteria by electroporation comprises:
preparing a suspension of bacteria in a solution of desired DNA, the desired DNA comprising a marker gene and other desired genes;
placing the suspension of bacteria in a container, the suspension in contact with a first and a second electrode;
cooling the suspension of bacteria;
applying a voltage burst between the first electrode and the second electrode such that an electric field is applied to the suspension of bacteria to produce transformed bacteria, the voltage burst comprising an initial portion and a later portion, the initial portion having a total magnitude of not less than eleven kilovolts per centimeter of distance between the first and second electrode, the later portion having a DC component less than thirty percent of the total magnitude of the initial portion, and an AC component having peak-to-peak amplitude of greater than six and a half percent of the total magnitude of the initial portion;
wherein the AC component of the voltage burst has a frequency between three and one hundred twenty-five megahertz;
wherein the voltage burst has duration between three and twenty-five milliseconds;
culturing the transformed bacteria; and
selecting transformed bacteria.

2. The method of claim 1 wherein the initial portion of the voltage burst lasts less than twenty percent of the duration of the voltage burst.

3. The method of claim 1 wherein the AC component of the voltage burst frequency is within twenty percent of about twenty-four megahertz.

4. The method of claim 3 wherein the AC component of the voltage burst overlaps the initial portion.

5. The method of claim 4 wherein the selecting of transformed bacteria is performed by performing the culturing of transformed bacteria on media containing at least one antibiotic for which transformed bacteria are resistant and for which non-transformed bacteria are susceptible.

6. The method of claim 4 wherein the cooling of the suspension of bacteria is performed by placing part of the container in an ice block.

7. The method of claim 1 wherein the bacteria are selected from those bacteria capable of forming endospores.

8. The method of claim 7 wherein the bacteria are selected from the group of bacteria consisting of genus Clostridium, genus Thermoanaerobacterium and genus Thermoanaerbacter.

9. The method of claim 8 wherein the AC component of the voltage burst frequency is within twenty percent of about twenty-four megahertz.

10. The method of claim 9 wherein the AC component of the voltage burst overlaps the initial portion.

11. The method of claim 10 wherein the selecting of transformed bacteria is performed by performing the culturing of transformed bacteria on media containing at least one antibiotic for which transformed bacteria are resistant and for which non-transformed bacteria are susceptible.

12. The method of claim 11 wherein the voltage burst has a total duration of eight to ten milliseconds.

13. A method for transforming bacteria by electroporation comprises:
preparing a suspension of bacteria in a solution of desired DNA, the desired DNA comprising a marker gene and other desired genes;
placing the suspension of bacteria in a container with a first and a second electrode, and connecting a current limiting device in series with the second electrode cooling the suspension of bacteria;
applying a voltage burst between the first electrode and the current limiting device to the suspension of bacteria such that an electric field is applied to the suspension of bacteria to produce transformed bacteria, the voltage burst having a total magnitude between eleven kilovolts and twenty-five kilovolts per centimeter of distance between the first and second electrode, the burst having an AC component having a peak-to-peak magnitude of greater than six and a half percent of the total magnitude and a DC component greater than the magnitude of the AC component;
wherein the AC component of the voltage burst has a frequency between three and one hundred twenty-five megahertz;
wherein the voltage burst has duration between three and twenty-five milliseconds;
culturing the transformed bacteria; and
selecting transformed bacteria.

14. The method of claim 13 wherein the AC component of the voltage burst frequency is within twenty percent of about twenty-four megahertz.

15. The method of claim 14 wherein the selecting of transformed bacteria is performed by culturing of transformed bacteria on media containing at least one antibiotic for which transformed bacteria are resistant and for which non-transformed bacteria are susceptible.

16. The method of claim 13 wherein the cooling of the suspension of bacteria is performed by placing part of the container in an ice block.

17. The method of claim 13 wherein the bacteria are selected from those bacteria capable of forming endospores.

18. The method of claim 17 wherein the bacteria are selected from the group of bacteria consisting of genus Clostridium, genus Thermoanaerobacterium and genus Thermoanaerbacter.

19. The method of claim 18 wherein the AC component of the voltage burst frequency is within twenty percent of about twenty-four megahertz.

20. Apparatus for transforming bacteria by electroporation comprising:
an electroporation cuvette comprising a container for a bacterial suspension in a DNA solution, the cuvette further comprising a first and a second electrode separated by an electrode spacing distance; and
a burst generator for generating an electrical burst between the first electrode and the second electrode, the generated electrical burst having a DC component and an AC component, the burst having a peak magnitude between eleven kilovolts and twenty-five kilovolts per centimeter of spacing distance between the first and second electrodes for at least five percent of a pulsewidth of the burst, the pulsewidth of the burst between three and twenty-five milliseconds, the electrical burst further comprising an AC component having a frequency of between three megahertz and one hundred twenty-five megahertz and having an amplitude greater than six and a half percent of the peak magnitude of the burst.

21. The apparatus of claim 20 wherein the DC component of the burst comprises an initial portion and a later portion wherein the later has a DC component of between ten and thirty percent of the total magnitude of the initial portion

22. The apparatus of claim 20 wherein the AC component has a frequency of within about twenty percent of twenty-four megahertz.

23. A method for transforming bacteria by electroporation comprises:
preparing a suspension of bacteria in a solution of desired DNA, the desired DNA comprising a marker gene and other desired genes;
placing the suspension of bacteria in a container with a first and a second electrode, and connecting a current limiting device in series with the second electrode cooling the suspension of bacteria;
applying a voltage burst between the first electrode and the current limiting device to the suspension of bacteria such that an electric field is applied to the suspension of bacteria to produce transformed bacteria, the voltage burst comprising a superposition of a first and a second AC signal and having a total magnitude of not less than eleven kilovolts per centimeter of distance between the first and second electrode, the second AC component having peak-to-peak amplitude of between than six and thirty percent of the total magnitude;
wherein the second AC component of the voltage burst has a frequency between three and one hundred twenty-five megahertz;
wherein the first AC component of the voltage burst has a frequency between twenty kilohertz and five hundred kilohertz.
wherein the voltage burst has duration between three and twenty-five milliseconds;
culturing the transformed bacteria; and
selecting transformed bacteria.

24. A method for transforming bacteria by electroporation comprises:
preparing a suspension of bacteria in a solution of desired DNA, the desired DNA comprising a marker gene and other desired genes;
placing the suspension of bacteria in a container, the suspension in contact with a first and a second electrode;
cooling the suspension of bacteria;
applying a voltage burst between the first electrode and the second electrode such that an electric field is applied to the suspension of bacteria to produce transformed bacteria, the voltage burst comprising an AC signal having a frequency between three megahertz and one hundred twenty-five megahertz, the AC signal comprising at least six percent of a total magnitude of the voltage burst;
wherein the voltage burst has duration between three and twenty-five milliseconds;
culturing the transformed bacteria; and
selecting transformed bacteria.

25. The method of claim 24 wherein the AC signal is within about twenty percent of twenty-four megahertz.

26. The method of claim 24 wherein the voltage burst has at least an initial portion that has a magnitude that exceeds eleven kilovolts per centimeter of a spacing between the first and second electrodes.

27. The method of claim 26 wherein the voltage burst has a later portion following the initial portion that has a magnitude less than thirty percent of the magnitude of the initial portion, and the later portion comprises at least half the burst length.

28. The method of claim 27 wherein the voltage burst has at least an initial DC component having a magnitude that exceeds eleven kilovolts per centimeter of a spacing between the first and second electrodes.

29. The method of claim 28 wherein the bacteria are selected from the group of bacteria consisting of genus Clostridium, genus Thermoanaerobacterium and genus Thermoanaerbacter.

30. The method of claim 24 wherein the voltage burst has a second AC component in having a frequency between twenty kilohertz and five hundred kilohertz, and where the second AC component has magnitude greater than the AC signal.

31. The method of claim 30 wherein the bacteria are selected from the group of bacteria consisting of genus Clostridium, genus Thermoanaerobacterium and genus Thermoanaerbacter

32. The method of claim 31 wherein the selecting of transformed bacteria is performed by performing the culturing of transformed bacteria on media containing at least one antibiotic for which transformed bacteria are resistant and for which non-transformed bacteria are susceptible.

33. Apparatus for performing electroporation comprising:
a container capable of holding a bacterial suspension in a DNA solution in contact with a first and a second electrode; and
apparatus for applying a voltage burst between the first electrode and the second electrode, the voltage burst comprising an AC signal having a frequency between three megahertz and one hundred twenty-five megahertz, the AC signal comprising at least six percent of a total magnitude of the voltage burst;
wherein the voltage burst has duration between three and twenty-five milliseconds; and
wherein the voltage burst has at least an initial portion that has a magnitude that exceeds eleven kilovolts per centimeter of a spacing between the first and second electrodes.

34. The apparatus of claim 33 wherein the voltage burst has a later portion following the initial portion that has a magnitude less than thirty percent of the magnitude of the initial portion, and the later portion comprises at least half the burst length.

35. The apparatus of claim 33 wherein the voltage burst has at least an initial DC component having a magnitude that exceeds eleven kilovolts per centimeter of a spacing between the first and second electrodes.

36. The apparatus of claim 35 wherein the voltage burst has a later portion following the initial portion that has a magnitude less than thirty percent of the magnitude of the initial portion, and the later portion comprises at least half the burst length.
